(19) United States
(12) Patent Application Publication
(10) Pub. No.: US 2004/0076610 A1
(43) Pub. Date: Apr. 22, 2004

(54) Method of treating portal hypertension

(75) Inventor: Don C. Rockey, Chapel Hill, NC (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY

(21) Appl. No.: 10/611,432

(22) Filed: Jul. 2, 2003

Related U.S. Application Data
(63) Continuation of application No. 09/805,440, filed on Mar. 14, 2001, now abandoned.
(60) Provisional application No. 60/189,088, filed on Mar. 14, 2000.

(51) Int. Cl. A61K 48/00

(52) U.S. Cl. 424/93.2; 514/44

(57) ABSTRACT

The present invention relates, in general, to portal hypertension and, in particular, to a method of reducing portal hypertension using an isoform of nitric oxide synthase.

This application claims priority from U.S. Provisional Application No. 60/189,088, filed Mar. 14, 2000, the entire content of which is incorporated herein by reference.

### TECHNICAL FIELD

The present invention relates, in general, to portal hypertension and, in particular, to a method of reducing portal hypertension using an isoform of nitric oxide synthase (NOS).

### BACKGROUND

The common result of many types of chronic liver injury is cirrhosis, which leads to increased intrahepatic resistance and portal hypertension (Friedman, N. Engl. J. Med. 328:1828-1835 (1993). Portal hypertension in turn has profound clinical consequences, many of which are associated with substantial morbidity and mortality. The pathologic basis of portal hypertension is complex and involves multiple factors (Shah et al, Hepatology 27(1):279-288 (1998)). However, in most instances an increase in intrahepatic resistance to blood flow is an early and critical component. Recent evidence links perisinusoidal stellate cells (also known as Ito cells or lipocytes), which are analogous to tissue pericytes or vascular smooth muscle cells, to a role in portal hypertension via their capacity to regulate blood flow within the liver by contraction and constriction of sinusoids (Bauer et al, Am. J. Physiol. 267:G143-G149 (1994), Zhang et al, Am. J. Physiol. 266:G624-G632 (1994), Bauer et al, Hepatology 22 (5): 1565-1576 (1995), Okumura et al, Hepatology 19:155-161 (1994), Suematsu et al, J. Clin. Invest. 96 (5): 2431-2437 (1995)).

In the injured liver, stellate cells undergo a striking functional transition termed “activation”. A critical feature of activation is the acquisition by stellate cells of smooth muscle proteins, including smooth muscle isoforms of actin and myosin (Tanaka et al, J. Pathol. 164:273-278 (1991)). This transition is associated with an enhanced contractile phenotype; as a result of activation, stellate cell contractility is greatest in the injured liver (Bauer et al, Hepatology 22 (5): 1565-1576 (1995), Rockey et al, J. Clin. Invest. 92:1795-1804 (1993)). Recent data indicate that the family of endotheline are potent stimulators of stellate cell contraction while vasorelaxing substances including nitric oxide (NO) counterbalance the contractile response. Further, in the injured liver, ET-1 production is increased (as a result of enhanced production by stellate cells (Pinzani et al, Gastroenterology 110 (2): 534-548 (1996), Gandhi et al, Life Sci. 58 (1): 55-62 (1996), Rockey et al, Hepatology 27 (2): 472-480 (1998))) and in addition, NO release from sinusoidal endothelial cells is reduced, ostensibly due to impaired function of endothelial cell NOS (ecNOS) (Shah et al, J. Clin. Invest. 100 (11):2923-2930 (1997), Rockey et al, Gastroenterology 114 (2):344-351 (1998), Gupta et al, Hepatology 28 (4): 926-931 (1998), Shah et al, Gastroenterology 117:1222-1228 (1999)). These data indicate that the dynamic balance of ET-1 and NO is abnormal in cirrhosis, favoring sinusoidal and possibly vascular wall constriction by perisinusoidal stellate cells and perhaps by hepatic vascular smooth muscle cells, respectively. Further, the data suggest that on a cellular level, anomalous production of each ET-1 and NO in the injured liver contribute to elevated intrahepatic resistance and portal hypertension.

The present invention results, at least in part, from studies demonstrating efficient adenovirus (Ad)-mediated transduction of neuronal NOS (nNOS) in liver sinusoidal (each sinusoidal endothelial and stellate) cells as well as in hepatocytes both in vitro and in vivo. Expression of nNOS in each cell type leads to enhanced NO release, an effect that results in inhibition of stellate cell contraction. The present invention provides a method of significantly reducing portal pressure and intrahepatic resistance to flow.

### SUMMARY OF THE INVENTION

The present invention relates to a method of reducing portal hypertension. The method comprises administering to a patient in need of such reduction the neuronal NO synthase isoform.

Objects and advantages of the present invention will be clear from the description that follow.

### BRIEF DESCRIPTION OF THE DRAWINGS

FIGS 1A-1E. Adenovirus-mediated n-NOS Gene Delivery and Activity of nNOS. Parenchymal and non-parenchymal cells were prepared as in the Examples. After isolation, cells were grown for 48-72 hours under standard conditions and then transduced with adenovirus containing β-gal (Ad.β-gal) or nNOS (Ad.nNOS). In FIG. 1A and FIG. 1B, cells were transduced with Ad.β-gal or Ad.nNOS, respectively and stained for NADPH diaphorase. In FIG. 1C, the transduction efficiency after exposure to Ad.nNOS was determined by cell counting (Ad.β-gal is used as an additional control). In FIG. 1D, cell lysates (25 μg total protein) were immunoblotted for nNOS. In FIG. 1E, 24 hours after transduction, culture supernatants were collected and nitrite detected. *P<0.01 compared to control or either Ad.β-gal for each cell type, (n=3). Abbreviations: H=hepatocyte; SEC=sinusoidal endothelial cell; HSC=hepatic stellate cell.

FIGS. 2A-2C. nNOS Transduction In Vivo in Normal and Injured Hepatic Cells. Liver injury (bile duct ligation or carbon tetrachloride administration) was induced. Adenovirus (1.5×10¹¹ pfu/kg via femoral vein) was administered to normal rats or to injured rats two days after bile duct ligation or the final dose of carbon tetrachloride. Hepatocytes, sinusoidal endothelial cells and stellate cells were isolated 7 days after adenovirus administration and lysed immediately in sample buffer. In FIG. 2A is shown a representative (of 4) immunoblot (25 μg total protein) probing cells isolated from normal liver. In FIG. 2B and FIG. 2C are shown immunoblots of cells isolated from bile duct ligated and carbon tetrachloride treated animals, respectively. H=hepatocyte; SEC=sinusoidal endothelial cell; HSC=hepatic stellate cell

FIGS. 3A and 3B. Effect of nNOS Gene Transfer on Stellate Cell Contractility In Vitro and In Vivo. In FIG. 3A, the effect of transduced nNOS on stellate cell contractility in vitro was determined. Stellate cells from normal livers were isolated, placed on thick collagen lattices and allowed to undergo spontaneous activation. Cells were transduced with Ad.nNOS and 24 hours later contraction assays were performed. In FIG. 3B, bile duct ligation was performed and adenovirus containing nNOS or β-galactosidase was administered 2 days later; stellate cells were isolated and placed on thick collagen matrices. After adherence (for 18 hours), lattices containing cells were placed in serum free medium after which endothelin-1 was added at the indicated concentrations and lattices were detached and contraction was measured for a further 24 hours. The open bars represent cells from control animals (i.e., vehicle alone), the crossed bars represent cells from rats receiving Ad.β-gal and the closed bars depict cells from animals receiving Ad.nNOS. * P<0.01 compared to control or Ad.β-gal (n=3); ** P<0.001 compared to control or Ad.β-gal (n=3).

FIG. 4. Paracrine effect of transduced nNOS on stellate cell contractility. Stellate cells from normal livers were isolated, placed on thick collagen lattices and allowed to undergo spontaneous activation. Adenovirus (1.5×10¹¹ pfu/kg via femoral vein) containing nNOS or β-gal was administered to normal rats and hepatocytes were isolated 4 days later. Hepatocytes were then layered on top of stellate cells and contraction was induced by serum (20%) 24 hours later. * P<0.01 compared to control or Ad.β-gal (n=3).

FIGS. 5A-5C. Adenovirus-mediated gene delivery results in high-efficiency gene transfer in primary cultured parenchymal and non-parenchymal hepatic cells.

### DETAILED DESCRIPTION OF THE INVENTION

The present invention provides a therapy for portal hypertension caused, for example, by cirrhosis.

The studies described in the Examples that follow establish that adenovirus readily leads to gene transfer not only of hepatocytes, as has been previously established (Kovesdi et al., Curr. Opin. Biotechnol. 8(5):583-589 (1997), Maher et al, Hepatology 25(3):624-630 (1997), but also of sinusoidal endothelial cells and stellate cells, the latter consistent with a previous report that demonstrated efficient in vitro transduction of cultured stellate cells (Hellerbrand et al, Hepatology 27(5):1285-1295 (1998)). The transduction efficiency of sinusoidal endothelial and stellate cells in vivo was high. This result has not been previously described but is consistent with the fact that hematogenous compounds must traverse the sinusoidal endothelium as well as the sinusoidal space of Disse (wherein reside stellate cells) prior to coming into contact with hepatocytes. In addition, the studies described in the Examples demonstrate that adenovirus transduced all cell types in vivo even after profound liver injury (Kountouras et al, Br. J. Exp. Pathol. 65:305-311 (1984), Proctor et al, Gastroenterology 83:1183-1190 (1982)). Although the transduction efficiency of each hepatocytes and nonparenchymal cells was reduced after injury, it remained of high magnitude and resulted in prominent NOS activity in the injured liver. This finding makes possible gene therapy in diseased liver as well as in other parenchymal organs that have been injured.

While the studies described in the Examples are based on the use of an adenoviral gene transfer system, the invention is not limited to the use of adenoviruses. Indeed, any of a variety of gene therapy vectors can be used, including adenoviral vectors, retroviral vectors and adeno-associated viral vectors, as can DNA-ligand complexes. Optimum vectors can be selected so as to allow for, for example, regulable expression (e.g., high or low levels or short or long lived), cell specificity, organ specificity, efficient gene transfer, nonimmunogenicity and nonhepatotoxicity. Vectors can also be selected to facilitate delivery of large constructs.

As regards adenoviral vectors, these vectors have been very popular because of the fact that adenoviral transduction leads to high level gene expression, and, in addition, adenoviruses efficiently transduce the majority of cell types, including those that are not dividing. Standard, or first-generation adenoviruses were constructed by removing essential viral genes (E1a and E1b) from the viral genome and replacing the deleted viral genes with the gene to be expressed. Among the best-characterized adenoviruses are types 2 and 5. These adenoviruses have substantial tropism for epithelial cells from the respiratory tract as well as the liver. Because of the tropism for the liver, simple intravenous administration of adenoviral vectors results in high-level expression in liver cells. An important consideration in the selection of adenoviral vectors is the fact that gene expression is transient, perhaps lasting for no more than a few weeks at maximum. The mechanism for this transient time period of gene expression appears to be that the virus remains episomal in location, and that immune mediated eradication of infected cells is prominent. Additionally, the mixed humoral and T-cell mediated immune response (against adenoviral cellular proteins), may preclude repeat administration of adenovirus. Finally, administration of high titers of virus can lead to significant hepatotoxicity.

Retroviruses, by comparison, integrate into the host genome, are transmitted to progeny cells, and, therefore, induce long lasting gene transfer. The most widely used (murine) retroviral vector systems require two major components. First, essential viral genes are removed (in order to allow room for the transgene, as well as to remove viral DNA sequences required for independent viral replication). A transfer vector for the transgene carrying cis-acting elements necessary for replication and efficient integration of retroviral DNA is thus created. Subsequently, this vector is transfected into a “packaging” cell line that produces trans-acting proteins necessary for structural and catalytic functions of the virus; this results in the genesis of “producer cells” which produce mature virions containing the gene of interest. The advantage of long-lasting gene transfer and expression by retroviruses may offset by the fact that retroviruses are unable to infect non-dividing cells. In addition, integration of retroviruses may activate latent oncogenes, and, in addition, may interfere with normal gene function (i.e., inactivate tumor suppressor or other critical genes). However, advances in design of retroviral vectors have resulted in greater degree of biosafety, expanded host range, and increased stability of the virus particles. To date, retroviral vectors have been used predominantly in ex vivo gene therapy protocols, although in vivo gene therapy has been achieved after the liver cells have been stimulated to regenerate.

Lentiviruses are a class of complex retroviruses, the best known of which is the human immunodeficiency virus type 1 (HIV-1). A major advantage of this class of vectors is that they are able to infect, and, therefore, transduce, nondividing cells. Currently used lentiviral vectors are derived from HIV-1, and have an excellent biosafety profile. A potential drawback of these vectors (as with retroviruses) is their random integration into the recipient genome, leading to concerns that integration may activate oncogenes or inactivate tumor suppressor genes.

As indicated above, adeno-associated viral vectors are also suitable for use in the invention. Adeno-associated virus (AAV) is a human naturally occurring, replication-defective, parvovirus. It is similar to hepatitis D virus in this regard. This virus can integrate in a site-specific fashion into a transcriptionally silent area of chromosome 19 (McKeon et al, Hum. Gene Ther. 7:1615 (1996); Young et al, J. Virol. 74:3953 (2000)), can integrate into non-dividing cells, and is not infectious. It appears that while wild-type viruses can integrate specifically into chromosome 19, recombinant viruses appear to integrate randomly.

Nonviral gene transfer and naked DNA vectors, while perhaps less preferred, can also be used in the present method. One technique that can be used is simply to attach a gene (e.g., the NOS gene) to a carrier. Suitable carriers include either polymer based cationic carriers (conjugates) or lipid based vectors (liposomes). In order to specifically target hepatocytes, advantage can be taken of the asialoglycoprotein receptor—which mediates receptor mediated endocytosis in hepatocytes (Chowdhury et al, J. Biol. Chem. 268:11265 (1993); Shetty et al, Gut 46:136 (2000); Wu et al, J. Biol. Chem. 266:14338 (1991)). Ligands recognized by the asialoglycoprotein receptor have been attached to genes of interest, typically in combination with a polycation such as polylysine. The DNA-polylysine-ligand complex binds to the asialoglycoprotein receptor and is taken up by receptor mediated endocytosis in hepatocytes. Naked DNA, can be used when transient and low level gene expression is acceptable (Chowdhury, Semin. Liv. Dis. 19:1 (1999); Shetty et al, Gut 46:136 (2000)).

In the studies described in the Examples, a heterologous NOS isoform, nNOS, was transduced. This isoform was selected in view of the fact that, to be functional, ecNOS must undergo extensive post-translational processing (including myristolation and disassociation from caveolae) in endothelial cells. Because post-translational handling defects may occur after liver injury, transduction with nNOS may be preferred, however, the invention includes within its scope the use of any NOS isoform.

The nucleic acid-containing compositions of the invention can be stored and administered, for example, in a sterile physiologically acceptable carrier, where the nucleic acid is dispersed in conjunction with any agents which aid in the introduction of the DNA into cells.

Various sterile solutions may be used for administration of the composition, including water, PBS, ethanol, lipids, etc. The concentration of the nucleic acid will be sufficient to provide a therapeutic dose, which will depend on the efficiency of transport into the cells.

Actual delivery of the gene sequence, formulated as described above, can be carried out by a variety of techniques including direct injection, intravenous injection and other physical methods. Administration can be by syringe needle, trocar, cannula, catheter, etc, as a bolus, a plurality of doses or extended infusion, etc.

The compositions containing the nucleic acid sequences can be administered for prophylactic and/or therapeutic treatments. In therapeutic application, compositions are administered to a patient in an amount sufficient to cure or at least partially arrest the condition and its complications. An amount adequate to accomplish this is defined as a “therapeutically effective dose” or “efficacious dose”. Amounts effective for this use will depend upon the severity of the condition, the general state of the patient, and the route of administration.

Certain aspects of the present invention are described in greater detail in the Examples that follow.

### EXAMPLES

The following experimental methods and details are referenced in the Examples that follow.

Animals and Liver Injury

Hepatic injury was induced in male retired breeder Sprague-Dawley rats (450-550 g) by bile duct ligation (BDL) for 9 days or by intragastric administration of carbon tetrachloride (CCl₄, administered at a concentration of 1 mL/Kg once per week for 10 weeks), each as previously described (Kountouras et al, Br. J. Exp. Pathol. 65:305-311 (1984), Proctor et al, Gastroenterology 83:1183-1190 (1982)).

Cell Isolation and Culture

Hepatocytes, stellate cells and sinusoidal endothelial cells were isolated as described previously (Bissell et al, J. Cell Biol. 59:722-734 (1973), de Leeuw et al, Hepatology 4:392-403 (1984), Friedman et al, Anal. Biochem. 161:207-218 (1987)). For nonparenchymal cells, after in situ perfusion of the liver with 20 mg % pronase (Boehringer Mannheim, Indianapolis, Ind.) followed by, collagenase (Crescent Chemical Co., Hauppauge, N.Y.), dispersed cell suspensions were layered on a discontinuous density gradient of 8.2% and 15.6% Accudenz (Accurate Chemical and Scientific, Westbury, N.Y.). The resulting upper layer consists of more than 95% stellate cells. Endothelial cells in the lower layer were further purified by centrifugal elutriation (18 ml/min flow) Hepatocytes isolated after collagenase perfusion (as above) were placed in modified medium 199 OR (Irving et al, Gastroenterology 87:1233-1247 (1984)) containing 5% fetal bovine serum (Gibco Lab., Grand Island, N.Y.); nonparenchymal cells were grown in the same medium containing 20% serum (10% horse/10% calf) (Flow Laboratories, Inc., Naperville, Ill.). The viability of all cells was verified by phase contrast microscopy as well as the ability to exclude propidium iodide. Cell viability of cultures utilized for study was greater than 95%.

Inhibition of NOS in Vivo

NOS was inhibited in vivo using 7-nitroindazole (7-NI, Sigma, St. Louis, Mo.), a selective nNOS inhibitor (Itzhak et al, Neuroreport 9(11):2485-2488 (1998)). 7-NI was dissolved in solution containing dimethyl sulfoxide/propylene glycol/distilled water (ratio of 1:3:6) and administered by intraperitoneal injection in a volume of 2.0 mL/kg. 7-NI was injected one day before bile duct ligation or the last dose of carbon tetrachloride at the dose of 25 mg/kg for a total of four doses.

Adenovirus Preparation

Ad.nNOS, containing a cDNA encoding rat neuronal NOS, driven by the cytomegalovirus immediate-early enhancer, was generated as previously described (Channon et al, Cardiovasc. Res. 32(5):962-972 (1996)). A recombinant adenovirus encoding β-galactosidase (Ad.β-gal), in the same cassette as Ad.nNOS, served as a control virus.

For cellular transduction with adenovirus, isolated cells were plated at a density of 1×10⁵ cells/mL in 35 or 60 mm plastic or collagen coated (hepatocytes and sinusoidal endothelial cells) culture and exposed to indicated concentrations of adenovirus for 2 hours at 37° C. Subsequently, adenovirus-containing medium was exchanged for standard medium. For in vivo experiments, rats were anaesthetized and Ad.nNOS or Ad.β-gal were administered via the femoral vein at the indicated titer. In these experiments adenovirus administration was timed to occur such that gene expression (i.e., 7-10 days after adenoviral injection) would be present at the time of cell harvest or physiologic harvest. For the bile duct ligation model, adenovirus was administered 2 days after bile duct ligation; for the carbon tetrachloride model, adenovirus was administered 2 days after the 10th and final dose of carbon tetrachloride. Cells were routinely harvested (and perfusion experiments performed) 7 days after adenovirus administration. In preliminary experiments, it was found that concentrations of 1.0×10¹⁰ pfu/kg to 6.0×10¹¹ pfu/kg effectively transduced high proportions of each parenchymal and stellate and endothelial cells, with small variation in transduction rate. Therefore, a concentration of 1.5×10¹¹ pfu/kg of Ad.nNOS was chosen to examine nNOS gene transfer. Heparinized blood was obtained from all rats at the time they were sacrificed.

NADPH-Diaphorase Activity, Immunohistochemistry

To determine transduction efficiency following Ad.nNOS exposure, NADPH-diaphorase activity were assessed. Cells were washed and fixed for 30 min followed by permeabilization with 0.3% Triton X-100 in PBS for another 30 min. Finally, cells were stained with 1.33 μl/mL nitroblue tetrazolium and 2 mg/ml NADPH in the same buffer for approximately 1 hour until the development of blue-purple staining was observed. Mock-infected cells were stained in parallel for the same period of time.

Immunohistochemistry to identify nNOS expression was performed as described (Chao et al, Methods Enzymol. 268:488-496 (1996)). Briefly, liver tissues were fixed n 4% buffered-paraformaldehyde, embedded in paraffin, sectioned (6-8 μm) and deoaraffinized by standard methods. After incubation of sections with 10% sheep serum in PBS with 0.1% Triton X-100, sections were incubated overnight with monoclonal anti-nNOS antibody (Transduction Laboratories, Lexington, Ky.), diluted 1:100. The first antibody was visualized using an avidin-biotin complex/alkaline phosphatase substrate detection method (Vector Laboratories, Burlingame, Calif.). Sections were lightly counterstained with Methyl Green (Vector Laboratories, Burlingame, Calif.), viewed and photographed with a Nikon TE 300 photomicroscope (Nikon CO., Tokyo, Japan).

Nitrite Determination

Nitrite levels were measured using the Griess assay as previously described (Green et al, Anal. Biochem. 126:131-138 (1982)). Nitrite concentrations determined in conditioned supernatants were normalized to total protein concentrations in the cell monolayer. In preliminary experiments, both nitrite and nitrate production were measured (the latter by enzymatically reducing nitrate to nitrite using nitrate reductase). Nitrate levels were approximately 30% of nitrate levels and these proportions remained highly consistent between experiments. Therefore nitrite alone was measured to confirm NOS activity and NO production.

Immunoblot

nNOS was detected by immunoblot with commercially available anti-nNOS (Transduction, Lab., Lexington, Ky.). In brief, freshly isolated cells or those in culture were washed twice with PBS and lysed with a solution containing 1% SDS, 1.0 mM sodium vanacate, 10 mM Tris pH 7.4. Proteins (25 μg total) were separated by SDS-PAGE and transferred to nitrocellulose. The membrane was incubated in 5% non-fat milk and then with anti-nNOS for 1 h at room temperature. nNOS was detected with anti-mouse IgG horseradish peroxidase-conjugated antibody followed by chemiluminescence (ECL Western blotting Analysis system, Amersham Life Science, Arlington Heights, Ill.). Bands were visualized on multiple exposures to autoradiography film (Eastman Kodak Co., Rochester, N.Y.) and data collected over a narrow range of X-ray film linearity and quantitated by scanning densitometry.

Stellate Cell Contractility

Stellate cell contractility was quantitated as described (Rockey et al, J. Clin. Invest. 92:1795-1804 (1993)). Briefly, culture vessels were pre-incubated with PBS containing 1% BSA for at least 1 hour at 37° C., then washed twice with PBS and air dried. A combination of Vitrogen (Celltrix Co., Santa Clara, Calif.), 10× minimal essential medium (MEM) (Gibco Lab., Grand Island, N.Y.) and 0.2 M HEPES (final collagen concentration of ≈2.4 mg/ml) is mixed at 4° C. After the solution hardened, cell suspensions were layered on top of formed lattices in standard culture medium. After a specified period of time in culture, serum free conditions are introduced, contractile mediators added and collagen lattices containing cells are detached by gentle circumferential dislodgment of the lattice using a micro-pipet tip. Contraction was monitored as change in lattice diameter (which is used to calculate area) over time.

Portal Pressure and Isolated Liver Perfusion

Portal pressure was measured in vivo by cannulating the portal vein with a polyethylene catheter (PE-50) and recorded with a strain gauge transducer. Subsequently, the liver was prepared as previously described and per-used with Krebs solution oxygenated with 95% O₂/5% CO₂ at 37° C. (Weisiger et al, J. Clin. Invest. 83:411-420 (1989)). The flow rate was adjusted to 0, 10, 20, 40, 50 ml/min in a pseudorandom order and the inlet pressure at zero flow (P_{Q=0}) and multiple-point P-Q relationships were generated as described (Pannen et al, Am. J. Physiol. 271(5 Pt 2):H1953-H1961 (1996)). The slope of the P-Q relationship (Slope_{PQR}) reflects the flow-dependent incremental resistance of the portal system.

Statistics

Analysis of variance or the student's t test was used for statistical comparisons. Cells from different animals were used in each experiment. For calculation of mean values and statistical variation, “n” indicates the number of separate experiments. Error bars depict the SEM; absence of error bars indicates that the SEM was <1% of the mean, unless stated otherwise.

### EXAMPLE 1

Adenovirus-Mediated nNOS Expression in Hepatocytes, Sinusoidal Endothelial Cells and Stellate Cells

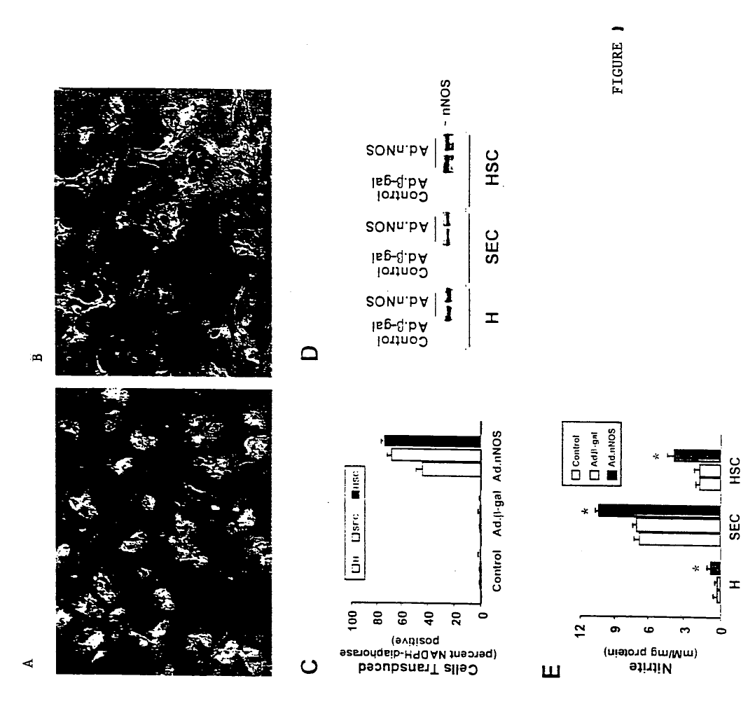

The ability of recombinant adenoviral vectors to transude hepatocytes, sinusoidal endothelial and stellate cells in vitro was first tested. Liver cells were isolated, cultured and incubated with 1×10⁹ pfu/mL Ad.β-gal. Although approximately 50% of hepatocytes were transduced at this titer, the transduction efficiency of sinusoidal endothelial and stellate cells was uniformly greater than 70% (not shown); as expected, transduction efficiency was directly dependent on adenoviral titer (not shown) for all cell types. Given the high level transduction with Ad.β-gal at a titer of 10⁹ pfu/mL, the same concentration was tested using the Ad.nNOS construct. Cultured cells were exposed to Ad.nNOS and after 24 hours, nNOS was detected by NADPH diaphorase staining (FIGS. 1A-1C); although all cell types expressed nNOS as assessed by NADPH diaphorase staining, the proportion of liver cells expressing NOS after transduction was highest in stellate cells (FIG. 1C). Further, stellate cells expressed abundant nNOS as established by immunoblot (FIG. 1D). Finally, NOS activity was confirmed by determination of nitrite concentrations in conditioned culture supernatants in hepatocytes, sinusoidal endothelial and stellate cells (FIG. 1E).

Adenovirus-Mediated nNOS Transduction in Normal and Injured Liver In Vivo

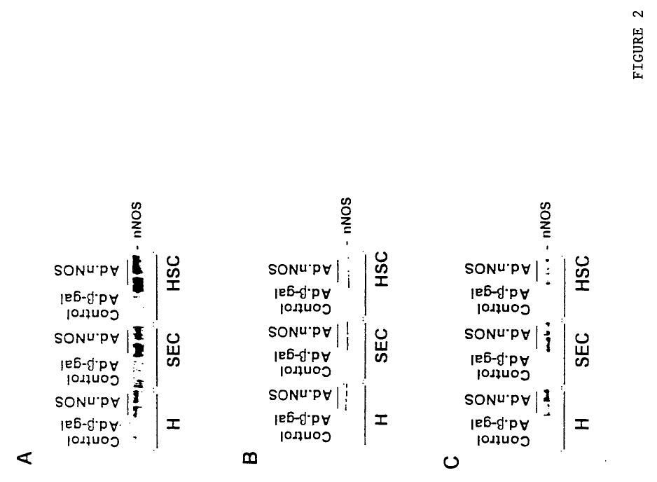

To determine the effectiveness of in vivo gene transfer of adenovirus, Ad.nNOS or Ad.β-gal (same titer) was administered via the femoral vein to normal rats or to those after liver injury (each bile duct ligation and carbon tetrachloride). nNOS expression was identified in the liver by immunohistochemical techniques as well as by quantitative methods (FIG. 2). nNOS was absent in livers transduced with control vector, whereas nNOS was prominent in cells lining sinusoids (consistent with either sinusoidal endothelial cells or stellate cell expression) and was also detected in hepatocytes and in vascular smooth muscle cells of portal veins. The global transduction rate of nNOS in the injured liver was less than that in normal livers. nNOS transduction in vivo with Ad.nNOS was more precisely examined by isolating cells from each normal and injured rat livers and in vivo gene therapy; nNOS was readily detectable in hepatocytes as well as sinusoidal endothelial and stellate cells (FIGS. 2A-2C), whether quantitated by immunoblot of isolated cells (FIGS. 2A-2C) or counting of NADPH-diaphorase positive isolated cells (Table 1). Transduction efficiency, as assessed by either method (as well as immunohistochemistry) was reduced after liver injury, but remained significant. Further, nitrite production, increased in all cell types after each form of liver injury, was less in cells from injured livers than in those from normal liver cells (Table 2). In the bile duct ligation model of injury, inducible nitric oxide synthase (iNOS) can be upregulated to a minor degree in sinusoidal endothelial and stellate cells, and can account for increased levels of nitrite in this situation (Rockey et al, Am. J. Physiol. 273 (1 Pt 1):G124-130 (1997)). Additionally, basal NOS activity was markedly decreased in sinusoidal endothelial cells after carbon tetrachloride induced liver injury, consistent with previous data (Rockey et al, Gastroenterology 114(2):344-351 (1998), Guota et al, Hepatology 28(4):926-931 (1998)). These data indicate that even after severe injury, Ad.nNOS transduced nNOS in injured hepatocytes, sinusoidal endothelial and stellate cells and led to increased NO production compared to control cells.

Transduced nNOS Reduces Stellate Cell Contractility

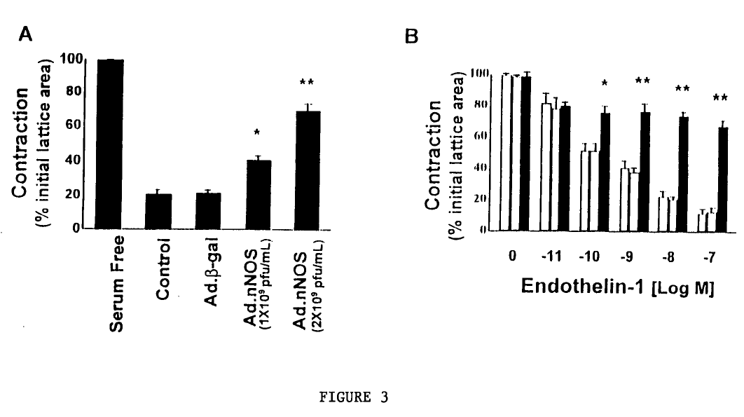

Given the data demonstrating transduction of nNOS in vitro and in vivo by Ad.nNOS, it was next determined whether transduced nNOS had biological effects on liver cells. Initially, the effect of nNOS derived NO on stellate cell contractility was evaluated in an in vitro assay. Stellate cells isolated from normal rats which had undergone spontaneous activation on collagen lattices were exposed to Ad.nNOS. Contraction of culture-activated stellate cells was significantly decreased in Ad.nNOS transfected cells compared to Ad.β-gal controls (FIG. 3A). Additionally, cells isolated from injured livers transduced with Ad.nNOS in vivo were also less contractile after exposure to endothelin-1 (2 nM) than those exposed to control Ad.β-gal (FIG. 3B).

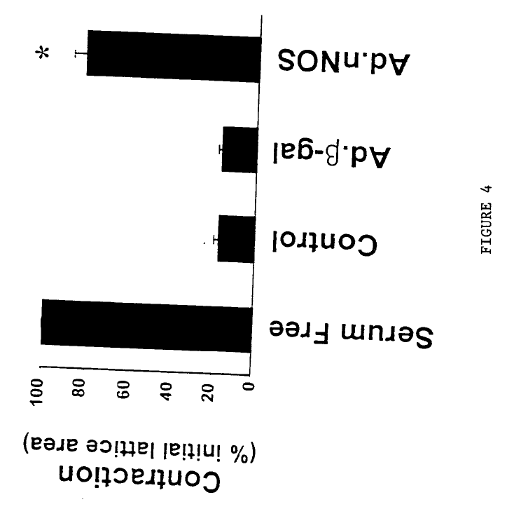

To test the possibility that NO produced in a paracrine fashion might also have effects on stellate cells, normal rats were transduced with Ad.nNOS (or as a control, Ad.β-gal) and the effect of transduced NO on contractility of activated stellate cells subsequently determined. NO generated from transduced hepatocytes co-cultured with activated stellate cells resulted in a 30-40% decrease in stellate cell contraction (FIG. 4). In hepatocyte-stellate cell co-cultures exposed to L-NMMA (100 μM), an inhibitor of NOS, the effect of nNOS was abrogated (not shown).

Transduced nNOS Reduces Portal Pressure and Intrahepatic Resistance after Cirrhosis and Portal Hypertension

To investigate the physiologic effect of transduced nNOS in the intact organism, portal pressure and intrahepatic resistance were measured in normal rats and those after liver injury (either bile duct ligation or carbon tetrachloride). In the bile duct ligation model, Ad.nNOS significantly reduced portal pressure in situ; portal pressure was 13.75±0.48 cm H₂O and 13.98±0.40 cm H₂O in control and Ad.β-gal treated animals, respectively, compared to 12.03±0.39 cm H₂O in Ad.nNOS treated animals (n≈4; all 1×10¹¹ pfu/kg; p<0.05 for control or Ad.β-gal compared to Ad.nNOS). Further, in the isolated perfused liver, the inlet pressure at zero flow (P_{Q=0}) was reduced in animals exposed to Ad.nNOS compared to either control or Ad.β-gal animals (Table 3). Similarly, in the carbon tetrachloride model of cirrhosis and portal hypertension, portal pressure in situ was reduced from 16.1±0.4 cm·H₂O and 16.0±0.2 cm·H₂O (control and Ad.β-gal, respectively) to 13.3±0.2 cm·H₂O (Ad.nNOS) (n=3). For this model, in the isolated perfused liver, P_{Q=0} was reduced in Ad.nNOS exposed animals compared to either control or Ad.β-gal exposed animals, while Slope_{PQR} was not significantly altered by nNOS transduction (Table 3). Since changes in Slope_{PQR} reflect predominantly pre-sinusoidal events while changes in P_{Q=0} reflect sinusoidal events (Pannen et al, Am. J. Physiol. 271(5 Pt 2):H1953-H1961 (1996)) , the data suggest that the effects of transduced Ad.nNOS are likely to be at the level of the sinusoid. Finally, in order to establish that the effect of nNOS was due to NO production, the effect of 7-nitroindazole, a selective nNOS inhibitor, was examined (Table 3). As expected, this inhibitor significantly abrogated the effect of Ad.nNOS.

### EXAMPLE 2

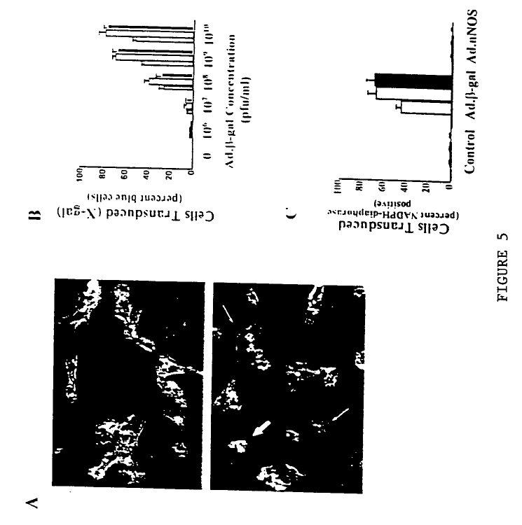

Parenchymal and non-parenchymal cells were isolated from normal rats. After isolation, cells were grown for 48-72 hours under standard conditions and then transduced with adenovirus containing β-gal (Ad.β-gal) or nNOS (Ad.nNOS). In FIG. 5A, stellate cells were exposed to Ad.nNOS (top) and to Ad.β-gal (bottom) [each, 5×10⁹ plaque-forming units (pfu)/mL for 2 hours]; each was incubated with beta-galactosidase. X-gal staining is absent from cells exposed to Ad.nNOS alone (similar results were obtained with control cells not exposed to adenovirus), but obvious in those exposed to Ad.β-gal. In the bottom panel the large arrow points to an untransduced cell with a clear cytoplasm, while the small arrows point to transduced cells with black cytoplasm. The refractile elements in the perinuclear region of the cells are retinoid droplets, characteristic of stellate cells. In FIG. 5B, the dose-response transduction efficiency of Ad.β-gal is shown. In FIG. 5C, detection of β-gal activity in controls (vehicle alone and Ad.nNOS) and after exposure to Ad.β-gal was determined by incubating cells with X-gal and cell counting. In FIG. 5B and FIG. 5C, open bars represent hepatocytes, hatched bars represent sinusoidal endothelial cells and closed bars, stellate cells.

All documents cited above are hereby incorporated in their entirety by reference.

One skilled in the art will appreciate from a reading of this disclosure that various changes in form and detail can be made without departing from the true scope of the invention.

What is claimed is:

1. A method of reducing portal hypertension in a patient in need of such reduction comprising administering to said patient a nucleic acid sequence encoding a polypeptide having nitric oxide synthase (NOS) activity under conditions such that said sequence is expressed and said reduction is thereby effected.

2. The method according to claim 1 wherein said sequence encodes neuronal NOS.

3. The method according to claim 1 wherein said sequence is present in a vector.

4. The method according to claim 3 wherein said vector is a viral vector.

5. The method according to claim 4 wherein said vector is an adenoviral, retroviral or adenoassociated viral vector.

6. The method according to claim 1 wherein said patient suffers from cirrhosis.

7. The method according to claim 1 wherein said nucleic acid is administered via the femoral vein of said patient.
